(19) United States
(12) Patent Application Publication
(10) Pub. No.: US 2024/0087682 A1
(43) Pub. Date: Mar. 14, 2024

(54) FACILITATION OF APTAMER SEQUENCE DESIGN USING ENCODING EFFICIENCY TO GUIDE CHOICE OF GENERATIVE MODELS

(71) Applicant: X Development LLC, Mountain View, CA (US)

(72) Inventors: Jon Deaton, Mountain View, CA (US); Hayley Weir, Mountain View, CA (US); Ryan Poplin, Newark, CA (US); Ivan Grubisic, Oakland, CA (US)

(73) Assignee: X Development LLC, Mountain View, CA (US)

(21) Appl. No.: 17/932,153

(22) Filed: Sep. 14, 2022

(51) Int. Cl. G16B 40/00; G16B 50/50

(52) U.S. Cl. CPC G16B 40/00; G16B 50/50

(57) ABSTRACT

A multi-dimensional latent space (defined by an Encoder model) corresponds to projections of sequences of aptamers. An architecture of the Encoder model, a hyperparameter of the Encoder model, or a characteristic of a training data set used to train the Encoder model was selected using an assessment of an encoding-efficiency of the Encoder model that is based on: a predicted extents to which representations in an embedding space are indicative of specific aptamer sequences to which a probability distribution of the embedding space differs from a probability distribution of a source space that represents individual base-pairs; generating projections in the latent space using representations of aptamers and the Encoder model; identifying one or more candidate aptamers for the particular target using the projections and the Decoder model; and outputting an identification of the one or more candidate aptamers.

### BACKGROUND

Aptamers are short sequences of single-stranded oligonucleotides (e.g., anything that is characterized as a nucleic acid, including xenobases). The sugar backbone of the single-stranded oligonucleotides functions as the acid and the A (adenine), T (thymine), C (cytosine), G (guanine) refers to the base. An aptamer can involve modifications to either the acid or the base. Aptamers have been shown to selectively bind to specific targets (e.g., proteins, protein complexes, peptides, carbohydrates, inorganic molecules, organic molecules such as metabolites, cells, etc.) with high binding affinity. Further, aptamers can be highly specific, in that a given aptamer may exhibit high binding affinity for one target but low binding affinity for many other targets. Thus, aptamers can be used to (for example) bind to disease-signature targets to facilitate a diagnostic process, bind to a treatment target to effectively deliver a treatment (e.g., a therapeutic or a cytotoxic agent linked to the aptamer), bind to target molecules within a mixture to facilitate purification, bind to a target to neutralize its biological effects, etc. However, the utility of an aptamer hinges on a degree to which it effectively binds to a target.

Frequently, an iterative experimental process (e.g., Systematic Evolution of Ligands by EXponential Enrichment (SELEX)) is used to identify aptamers that are selectively bound to target molecules with high affinity. In the iterative experimental process, a nucleic acid library of oligonucleotide strands (aptamers) is incubated with a target molecule. Then, the target-bound oligonucleotide strands are separated from the unbound strands and amplified via polymerase chain reaction (PCR) to seed a new pool of oligonucleotide strands. This selection process is continued for a number (e.g., 6-15) rounds with increasingly stringent conditions, which ensure that the oligonucleotide strands obtained have the highest affinity to the target molecule.

The nucleic acid library typically includes 10¹⁴-10¹⁵ random oligonucleotide strands (aptamers). However, there are approximately a septillion (10²⁴) different aptamers that could be considered. Exploring this full space of candidate aptamers is impractical. However, given that present-day experiments are now only a sliver of the full space, it is highly likely that optimal aptamer selection is not currently being achieved. This is particularly true when it is important to assess the degree to which aptamers bind with multiple different targets, as only a small portion of aptamers will have the desired combination of binding affinities across the targets. Accordingly, while substantive studies on aptamers have progressed since the introduction of the SELEX process, it would take an enormous amount of resources and time to experimentally evaluate a septillion (10²⁴) different aptamers every time a new target is proposed. In particular, there is a need for improving upon current experimental limitations with scalable machine-learning modeling techniques to identify aptamers and derivatives thereof that selectively bind to target molecules with high affinity.

### SUMMARY

In some embodiments, a computer-implemented method is provided that includes: accessing a multi-dimensional latent space that corresponds to projections of sequences of aptamers, wherein the multi-dimensional latent space was defined by an Encoder model, wherein an architecture of the Encoder model, at least one hyperparameter of the Encoder model, or at least one characteristic of a training data set used to train the Encoder model was selected using an assessment of an encoding-efficiency of the Encoder model that is based on: a predicted extent to which representations in an embedding space are indicative of specific aptamer sequences; and a predicted extent to which a probability distribution of the embedding space differs from a probability distribution of a source space, wherein the source space represents individual base-pairs; generating a set of projections in the multi-dimensional latent space using representations of a plurality of aptamers and the Encoder model; identifying one or more candidate aptamers for the particular target using the set of projections and using the Decoder model, wherein the one or more candidate aptamers are a subset of the plurality of aptamers; and outputting an identification of the one or more candidate aptamers.

The selection of the architecture of the Encoder network, the at least one hyperparameter of the Encoder network, or the at least one characteristic of the training data set used to train the Encoder network may have been further based on a classification-performance metric corresponding to predictions of a Classifier model when different architectures, hyperparameters, or training sets were used to configure or train the Encoder network.

The extent to which a probability distribution of the embedding space may differ from a probability distribution of a source space includes a Kullback-Leibler distance.

The extent to which representations in an embedding space are indicative of specific aptamer sequences may be based on a reconstruction error relative to predictions of the Decoder model when different architectures, hyperparameters, or training sets were used to configure or train the Encoder network.

The method may include, prior to accessing the multi-dimensional latent space: selecting the architecture of the Encoder model based on: the predicted extent to which representations in an embedding space are indicative of specific aptamer sequences; and the predicted extent to which a probability distribution of the embedding space differs from the probability distribution of a source space.

The method may include, prior to accessing the multi-dimensional latent space: selecting the at least one hyperparameter of the Encoder model based on: the predicted extent to which representations in an embedding space are indicative of specific aptamer sequences; and the predicted extent to which a probability distribution of the embedding space differs from the probability distribution of a source space.

The method may include, prior to accessing the multi-dimensional latent space: selecting the at least one characteristic of a training data set of the Encoder model based on: the predicted extent to which representations in an embedding space are indicative of specific aptamer sequences; and the predicted extent to which a probability distribution of the embedding space differs from the probability distribution of a source space.

The architecture of the Encoder network may have been selected using the assessment of the encoding efficiency.

The at least one hyperparameter of the Encoder network may have been selected using the assessment of the encoding efficiency.

The at least one characteristic of the training data set of the Encoder network may have been selected using the assessment of the encoding efficiency. In some embodiments, a system is provided that includes one or more data processors and a non-transitory computer readable storage medium containing instructions which, when executed on the one or more data processors, cause the one or more data processors to perform part or all of one or more methods disclosed herein.

In some embodiments, a computer-program product is provided that is tangibly embodied in a non-transitory machine-readable storage medium and that includes instructions configured to cause one or more data processors to perform part or all of one or more methods disclosed herein.

The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention as claimed has been specifically disclosed by embodiments and optional features, modification and variation of the concepts herein disclosed may be resorted to by those skilled in the art, and that such modifications and variations are considered to be within the scope of this invention as defined by the appended claims.

### BRIEF DESCRIPTION OF THE DRAWINGS

The present disclosure is described in conjunction with the appended figures:

FIG. 1 illustrates an exemplary process for identifying using encoding efficiency variables to select a trained Encoder model to use to identify sequence candidates.

FIG. 2 shows a flowchart of an exemplary process 200 for using a trained Encoder model to identify aptamer sequences of interest.

FIG. 3 shows a block diagram of a pipeline 300 for strategically identifying and generating high affinity binders of molecular targets.

FIGS. 4A and 4B show the reconstruction error and encoding efficiency (respectively) for each of five models (with different hyperparameters) trained to identify functionally inhibiting Trypsin TNA aptamers.

In the appended figures, similar components and/or features can have the same reference label. Further, various components of the same type can be distinguished by following the reference label by a dash and a second label that distinguishes among the similar components. If only the first reference label is used in the specification, the description is applicable to any one of the similar components having the same first reference label irrespective of the second reference label.

### DETAILED DESCRIPTION

The ensuing description provides preferred exemplary embodiments only, and is not intended to limit the scope, applicability or configuration of the disclosure. Rather, the ensuing description of the preferred exemplary embodiments will provide those skilled in the art with an enabling description for implementing various embodiments. It is understood that various changes may be made in the function and arrangement of elements without departing from the spirit and scope as set forth in the appended claims.

Specific details are given in the following description to provide a thorough understanding of the embodiments. However, it will be understood that the embodiments may be practiced without these specific details. For example, circuits, systems, networks, processes, and other components may be shown as components in block diagram form in order not to obscure the embodiments in unnecessary detail. In other instances, well-known circuits, processes, algorithms, structures, and techniques may be shown without unnecessary detail in order to avoid obscuring the embodiments.

Also, it is noted that individual embodiments may be described as a process which is depicted as a flowchart, a flow diagram, a data flow diagram, a structure diagram, or a block diagram. Although a flowchart or diagram may describe the operations as a sequential process, many of the operations may be performed in parallel or concurrently. In addition, the order of the operations may be re-arranged. A process is terminated when its operations are completed, but could have additional steps not included in a figure. A process may correspond to a method, a function, a procedure, a subroutine, a subprogram, etc. When a process corresponds to a function, its termination may correspond to a return of the function to the calling function or the main function.

### Select Terminology

As used herein, the term “aptamer” refers to an oligonucleotide or peptide molecule. An aptamer scan be a single-stranded DNA or RNA (ssDNA or ssRNA) molecule. An aptamer may include (for example) less than 100, less than 80 or less than 60 nucleotides. Typically, a region of about 10-15 nucleotides of the aptamer is what binds to a target molecule. Thus, predicting that an aptamer will bind to a given target may include predicting that a particular portion of the aptamer will bind to a given target. Similarly, if it is predicted that a particular set of nucleotides (e.g., between 10-15 nucleotides) will bind to a given target, it may be predicted that a full aptamer that includes the particular set of nucleotide will bind to the given target. It will be appreciated that some disclosures herein may refer to generating a prediction or performing an assessment that pertains to one or more “aptamers”, though such disclosures may—more precisely—relate to prediction or performing an assessment that pertains to a binding region of an aptamer. (One or more full aptamers that includes the binding region may subsequently be identified and/or tested.)

As used herein, the term “binding affinity” refers to the free energy differences between native binding and unbound states, which measures the stability of native binding states (e.g., a measure of the strength of attraction between an aptamer and a target). As used herein, a “high binding affinity” is a result from stronger intermolecular forces between an aptamer and a target leading to a longer residence time at the binding site (higher “on” rate, lower “off” rate). The factors that lead to high affinity binding include a good fit between surface of the molecules in their ground state and charge complementary (i.e., stronger intermolecular forces between the aptamer and the target). These same factors generally also provide a high binding specificity for the targets, which can be used to simplify screening approaches aimed at developing strong therapeutic candidates that can bind the given molecular target. As used herein, the term “binding specificity” means the affinity of binding to one target relative to the other targets. As used herein, the term “high binding specificity” means the affinity of binding to one target is stronger relative to the other targets. Various aspects described herein design and validate aptamers as strong therapeutic candidates that can bind the given molecular target based on binding affinity. However, it should be understood that design and validation of aptamers could involve the assessment of binding affinity and/or binding specificity. Binding affinity can be measured or reported by the equilibrium dissociation constant (K_{D}), which is used to evaluate and rank order strengths of bimolecular interactions. The smaller the K_{D} value, the greater the binding affinity of the aptamer for its target. The larger the K_{D} value, the more weakly the target molecule and the aptamer are attracted to and bind to one another. In other words binding affinity and dissociation factor can have an inverse correlation. The strength of binding between an aptamer and its target can be also expressed by measuring or reporting a binding avidity between the aptamer and the target. While the term affinity characterizes an interaction between one aptamer domain with its binding site (assessed by corresponding dissociation constant K_{D}), the avidity refers to the overall strength of multiple binding interactions and can be described by the K_{D} of an aptamer-target complex.

### Overview

Identification of high affinity and high specificity binders (e.g., monoclonal antibodies, nucleic acid aptamers, and the like) of molecular targets (e.g., VEGF, HER2) has dramatically transformed treatment of many types of diseases (e.g., oncology, infectious disease, immune/inflammation, etc.). However, given the large search space of potential sequences (e.g., 10²⁴ or 4⁴⁰ potential sequences for the average aptamer or monoclonal antibody CDR-H3 binding loop) and the comparatively low-throughput of methodologies to assess the binding affinity of candidates (e.g., dozens to thousands per week), it is highly likely that optimal binder selection is not currently being achieved. While selection based approaches (e.g., phage display, SELEX, and the like) can potentially identify binders, among libraries of millions to trillions of candidates, there are several weaknesses with these approaches: (i) output is binary—it is challenging to know whether relatively strong binders in the library are actually strong binders; (ii) data is noisy—binding is dependent on every candidate encountering available target with the same relative frequency and variance from this can lead to many false negatives and some false positives; and (iii) capacity is much smaller than the total search space—phage display (max candidates ˜10⁹) and SELEX (max candidates ˜10¹⁴) search spaces much smaller than the total possible search space (additionally, it is generally difficult (or expensive) to characterize the portions of the total sequence space that are searched). Thus, two major challenges to identifying sequences (e.g., aptamers) of interest is that the quantity of sequences in a search space is very large, the signal-to-noise of initial labels may be typically very low, and yet—to improve the signal-to-noise, it can be important that diverse sequences are used for intermediate “experimental flywheel” testing so as to improve the signal to noise across multiple portions of the search space.

To address these challenges, efforts have been made to apply computational and machine-learning techniques in an “experiment in the loop” process to reduce the search space and design better binders. For example, the following computational and machine-learning techniques have been attempted to increase discovery of viable high affinity/high specificity binders of molecular targets: (i) identification of libraries more likely to bind via prediction from physics based models, (ii) input of selection data and design/identify more likely binders (for monoclonal antibodies and nucleic acid aptamers), and (iii) address other factors beyond affinity that affect commercialization and therapeutic potential. To date, however, these computational and machine-learning techniques have had limited success in designing markedly different sequences with better properties, let alone with sufficient predictive power to align on a small set of sequences appropriate for low-throughput characterization. Particularly, the techniques in the second category, often struggle to input sufficient data to identify or design candidates that are markedly different from the training sequences used to train the computation and machine-learning models.

To address these limitations and others, techniques and systems are disclosed herein to efficiently identify select sequences (e.g., aptamer sequences) to experimentally test to determine a binding affinity with a particular target. The percentages of molecules having the select sequences that bind to the particular target may be higher (when the selection is based on prioritizing target binding) as compared to a corresponding of a comparative subset of molecules selected based only on SELEX labels (given the high noise in SELEX labels). Alternatively, when aptamer selection is based on prioritizing no target binding, the percentage of the select aptamers that bind to the particular target may be lower as compared to a corresponding of a comparative subset of aptamers selected based only on SELEX labels.

More specifically, the techniques and systems may include implementations of processes that use machine-learning models to generate a projection for each of many sequences (e.g., aptamer sequences) and a filtering or traversal technique to select one or more sequences to experimentally investigate for binding affinity with a given target. It will be appreciated that many different types of models, model configurations, and/or training data sets may potentially be used to generate an embedded space. In some embodiments, an encoding-efficiency metric is used to convey an extent to which an embedding space retains (e.g., information of and/or potential complexities of) representations of data (from an original space) relative to an extent to which an embedding space to the original space. The encoding-efficiency metric may depend on (e.g., may include a numerator term that includes) an error (e.g., a reconstruction error that is based on outputs from a Decoder network that receives inputs from the Encoder network). The encoding-efficiency metric may be anti-correlated with the error, such that (for example) a term or numerator value is defined to be (1 minus the error) or an inverse of an error. A term or numerator that is anti-correlated with the error may further include (e.g., may be scaled by) a number of bits per position (e.g., bits per base or 2 bits per base) and/or an information unit-conversion from bits to nats to, e.g., the natural logarithm of 2, log(2). In this way, the numerator quantifies the amount of information required to encode, using the simplest possible encoding scheme, the bases of the sequence which the encoder/decoder networks reliably reconstruct correctly.

The encoding-efficiency metric may additionally or alternatively depend on the average amount of information that is encoded in the latent space by the Encoder network. For example, a term that represents the average information content of the latent embedding may be the Kullback-Leibler (KL) divergence which measures the difference between distribution the Encoder's generated probability distribution over latent variables and a prior distribution over the latent space variables. The KL divergence, in measuring the difference in these probability distributions, quantifies (in units of nats) the amount of information which is on average transmitted between encoder and decoder network via the latent embedding. The term that represents the average information content of the latent embedding produced by the encoder may appear in the denominator of the measure of encoding efficiency.

In some instances, the encoding-efficiency metric η is defined by Eqn. 1:

$$\\ \begin{matrix}η = \frac{( l - ϵ ) \cdot 2 \frac{bit}{base} \cdot log ( 2 ) \frac{nat}{bit}}{KL [ {q}_{ψ} ( 𝓏 \vert x ) ‖ p ( 𝓏 ) ]} & Eqn . 1\end{matrix}$$

where l is the sequence length, ε is the average reconstruction error (i.e., a number of bases incorrectly reconstructed by the decoder), p(z) is the prior distribution over latent variables, typically a zero-mean unit-variance gaussian, q_{φ}(z|x) is a distribution of input data in an embedding space generated by an Encoder network, and KL is a KL distance between the distributions. Specifically, the KL distance can be defined as:

$$\\ \begin{matrix}KL [ {q}_{ϕ} ( 𝓏 \vert x ) ‖ p ( 𝓏 ) ] = {\sum }_{i = 1}^{n} {σ}_{i}^{2} + {μ}_{i}^{2} - log {σ}_{i} - 1 & Eqn . 2\end{matrix}$$

where n is a number of dimensions in the latent space, and where μᵢ and σᵢ are the mean and standard deviation of the distribution of variables in the i-th dimension of the latent space generated by the Encoder network.

High KL distances indicate a lower degree of compression of a source space relative to that corresponding to low KL distances. For example, assuming that there are 4 potential base pairs at any position within a sequence, a 30-nucleotide sequences has 60 bits of information. The product of 60 bits and log(2) nats/bit is approximately 41.6 nats. Thus, a KL value that is greater than to 41.6 nats indicates that an Encoder network is representing sequences in a manner that is less efficient that directly encoding the sequence.

Because encoding-efficiency is a comparative measure of information, higher values of encoding-efficiency indicate a higher degree of compression of the source/sequence distributions. The encoding-efficiency metric η s configured to be equal to 1 when the embedding space encodes 2 bits per base that was correctly reconstructed and to be greater than 1 when the reconstruction results in a larger number of correctly identified bases than could be achieved with that amount of information and a direct encoding.

The encoding-efficiency metric may be used to (for example) select a type of Encoder network, to select parameters of a training data set, to evaluate a training data set, to evaluate a type of Encoder network, to select one or more hyperparameter values, to evaluate one or more hyperparameter values, to select a type of data (e.g., for training or implementation), to evaluate one or more types of data (e.g., for training or implementation, etc. Such selection and/or evaluation may further be based on one or more metrics. For example, another metric may indicate an extent to which a Decoder network can reliably reconstruct sequences using embeddings in the embedding space and/or an extent to which a property of peptides (e.g., a binding affinity) can be predicted based on representations of the peptides in the embedding space.

Therefore, the encoding-efficiency metric can be used (e.g., potentially in combination with one or more other metrics) to facilitate defining an embedding space that positions representations of sequences (e.g., aptamer sequences) in a manner that facilitates predicting how a particular property depends on sequences and that facilitates reconstructing which sequence corresponds to any particular point in the embedding space. Therefore, known labels and projections of corresponding sequences can be used to identify other sequences that are predicted to have a property of interest (e.g., a particularly high binding affinity for a particular target). In silico or laboratory experiments may then be performed to predict or experimentally observe the property of interest.

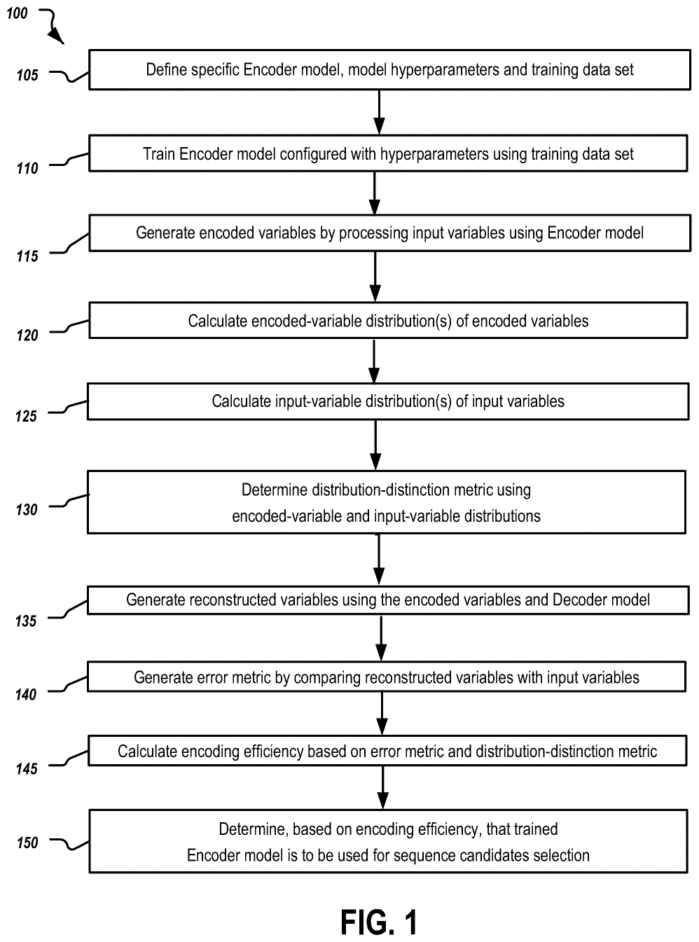

FIG. 1 illustrates an exemplary process 100 for identifying using encoding efficiency variables to select a trained Encoder model to use to identify sequence candidates. Block 105 includes defining a specific Encoder model, one or more specific model hyperparameters and a training data set. For example, block 105 may include identifying an LSTM model as the machine-learning model, defining a number of hidden layers as 2, setting a number of units in a dense layer to be 5, setting a dropout value to 0.5, and selecting a particular data set (e.g., a large SELEX data set as opposed to a smaller data set with labels generated based on elution experiments) that includes binding affinity data corresponding to a particular target as the training data set. The Encoder model may be or may include a neural network, such as a convolutional neural network, a Transformer, or a residual convolutional network.

In instances where the Encoder model does not have hyperparameters, block 105 need not include defining model hyperparameters. In some instances, hyperparameters are automatically defined (e.g., using an automated selection or tuning process). Defining an Encoder model may include specifying an architecture for the model. Defining an Encoder model, hyperparameter or training data set can include (for example) retrieving or selecting the Encoder model, hyperparameter or training data set.

The training data set may include multiple data elements, each of which can include an input data set that identifies or represents a sequence and that includes a label that indicates a property of a molecule coded by the sequence. For example, the label may identify a binding affinity between the molecule coded by the sequence and a particular target.

It will be appreciated that any of the model, hyperparameter or training data set definitions may be one of multiple options being explored.

At block 110, the Encoder model is configured with the hyperparameters and trained using the training data set. In some instances, the Encoder model is trained with one or more other models. For example, the Encoder model may be trained with a Decoder model configured to reconstruct full sequence representations from encoded variables in an embedding space and/or a Classifier model configured to predict a property of a molecule (e.g., a binding affinity with a particular target) based on encoded variables in the embedding space. Each of the Encoder network, the Decoder network, and the Classifier network may include or may be a neural network, such as a convolutional neural network, a Transformer, or a residual convolutional network. The Decoder network may include (for example) a neural network, such as auto-regressive Generator network that generates sequences incrementally through multiple passes.

At block 115, encoded variables are generated by processing input variables using the trained Encoder model. For example, the input variables may include representations of sequences in a first source space (e.g., which may be defined to have a separate dimension for each base pair, to represent all base pairs in a single dimension, to represent each individual base pair using one-hot encoding, etc.), and the encoded variables may include data points in an embedding space. In various instances, a dimensionality of a first source space is different than (e.g., smaller or larger than) that of the embedding space. However, a difference in dimensionalities is not necessarily predictive of a difference in encoding efficiencies, as (for example) any given dimension may be configured to be complex and may represent multiple base pairs.

At block 120, one or more encoded-variable distributions of the encoded variables are calculated. For example, a single distribution may be calculated across each dimension of the embedding space, or a multi-dimensional distribution can be calculated that corresponds to some or all of the dimensions in the embedding space.

At block 125, one or more input-variable distributions of the input variables (e.g., that were fed to the Encoder model at block 115) are calculated. For example, a single distribution may be calculated across each dimension of the source space, or a multi-dimensional distribution can be calculated that corresponds to some or all of the dimensions in the source space.

At block 130, a distribution-distinction metric is determined using the encoded-variable distribution and the input-variable distribution. The distribution-distinction metric may be a K-L distance (e.g., as defined in Eqn. 2). The distribution-distinction metric may be calculated based on a normalized or unnormalized degree of overlap between the two distributions in one or more dimensions. The distribution-distinction metric may be calculated based on a statistical term that assesses a degree of confidence that a given observable from one distribution was, in fact, part of the one distribution and not the other.

At block 135, a Decoder network (e.g., that was trained with the Encoder network at block 110) generates reconstructed variables using the encoded variables.

At block 140, an error metric e is generated by comparing the reconstructed variables with the input variables. The error metric can indicate an extent to which sequences identified in the reconstructed variables are the same as the corresponding sequences identified in the input variables. The error metric therefore depends on an extent to which an embedding space is sufficiently large or complex to capture differences between sequence representations and further on an extent to which there is structure in the input variables. If there is structure, then a good Encoder model should be able to represent the input variables in a space that is more compact than an input space. As a simple example, if every sequence in an input data set has a cytosine at a fourth position in a sequence, the fourth position potentially need not be represented at all in the embedding space, and training the Decoder network can result in reconstructions that nonetheless always include the correct base at this position.

At block 145, an encoding efficiency η is calculated based on the error metric and the distribution-distinction metric. The encoding efficiency η can represent an extent to which the embedding space efficiently captures information about the identity of base pairs in a sequence. The encoding efficiency η can be calculated using Eqn. 1.

At block 150, it is determined, based on the encoding efficiency, that the Encoder model trained with the training data set and configured with the hyperparameters is to be used to select sequence candidates. In some instances, blocks 105-145 are repeated for each of multiple Encoder models (e.g., having different architectures), for each of multiple hyperparameter sets and/or for each of multiple training data sets. A single Encoder model, single hyperparameter set, and/or single training data set can be selected at least in part by performing a comparison that involves the encoding efficiencies calculated for each iteration of blocks 105-145. The selection may further depend on (for example) the error metric generated at block 140 and/or a classification-performance metric c. The classification-performance metric c may be, may represent, or may depend on an error, a quantity of false positives, a quantity of false negatives, a quantity of true positives, and/or a quantity of true negatives generated based on known labels in the training data set and labels predicted by a Classifier network. In some instances, the labels are binary (e.g., indicating whether a molecule corresponding to a sequence bound to a particular target). In some instances, the labels are categorical or real numbers (e.g., identifying a binding affinity). To illustrate, an overall error may be defined to be:

E=w₁∈+w₂η+w₃c Eqn. 3

where w₁, w₂, and w₃ are weights. Block 150 may then include identifying the trained Encoder model associated with the lowest overall error (or with an overall error that is below a predefined threshold).

The weights may be selected by a user. For example, a value selected for the w₃ weight applied to the classification-performance metric c may depend on how resource-intensive a subsequent screening action is, a distribution of labels in the training data set, etc. To illustrate, if the labels are binary and 50% of the training data set was associated with one label (and the other 50% with the other label), the w₃ weight may be lower given that there is a relatively high likelihood sequences selected have a label of interest (e.g., as compared to an instance where the training data indicates that only 0.5% of data has a label of interest).

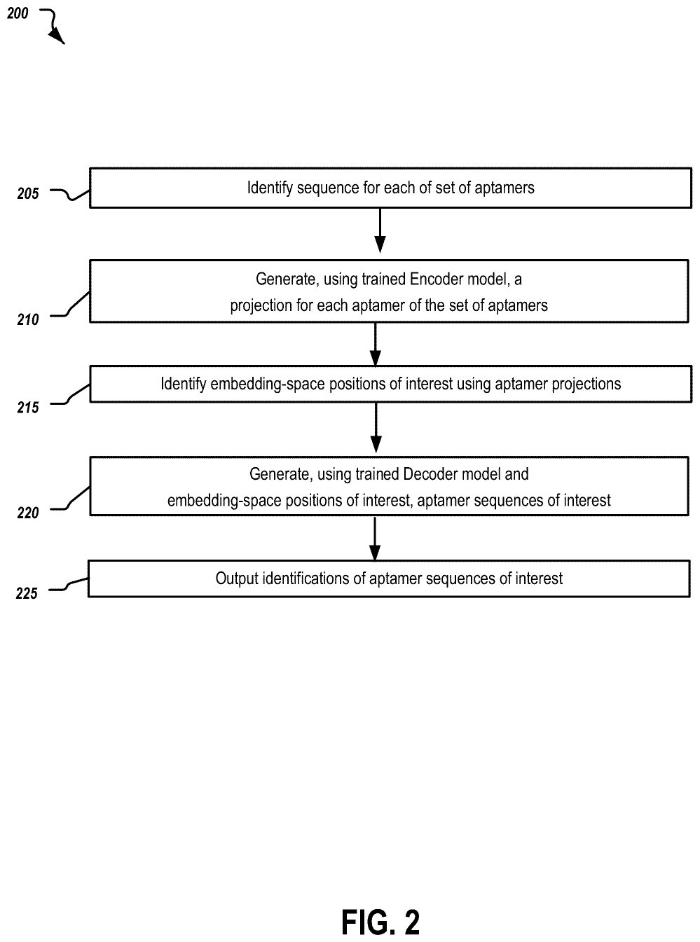

FIG. 2 shows a flowchart of an exemplary process 200 for using a trained Encoder model to identify aptamer sequences of interest. Process 200 begins at block 205, where a sequence for each aptamer in a set of aptamers is identified. The set of aptamers may include (for example) each and every aptamer represented in a given library, each and every aptamer represented in a given library and complying with a given condition (e.g., sequence length and/or user-specified attribute), a representative sample from a library, etc. The library may include a SELEX library. In some instances, the set of aptamers include a first plurality of aptamers, where each aptamer of the first plurality is associated with SELEX data indicating whether the aptamer is predicted to bind with a particular target and/or may include a second plurality of aptamers for which there is not SELEX data available (e.g., to a given user or developer) in relation to the particular target.

At block 210, for each of the set of aptamers, a projection for the aptamer in an embedding space can be generated using the trained Encoder model. Thus, the trained Encoder model can include the trained Encoder model for which it was determined to be used for selection of sequence selection using process 100.

At block 215, one or more embedding-space positions are identified using the aptamer projections. Labels assigned to one or more aptamers can be used for the identification. In instances where the Encoder model was trained in combination with a Classifier model, the labels may have been of a same type and/or may include labels that were used in the training. The labels may identify (for example) a binding affinity, binding probability, etc. with regards to the particular target. In some instances, the labels used to identify the embedding-space position(s) of interest are relatively noisy labels (e.g., SELEX labels).

In some instances, block 215 includes generating clusters using the projections of the set of aptamers and then identifying a subset of the clusters based on the labels, where the embedding-space positions of interest are the positions of aptamers assigned to the subset of the clusters. A cluster-specific binding metric can be generated for each cluster based on the labels of specific aptamer representations assigned to the cluster. For example, a cluster-specific binding metric may include a percentage of the aptamers assigned to the cluster that have an aptamer-specific metric of 1 or that have an aptamer-specific metric that exceeds a predefined threshold. As another example, a cluster-specific binding metric may include an average, median, standard deviation, or variance of the aptamer-specific metrics of aptamers assigned to the cluster. The subset of the set of clusters can be selected based on the cluster-specific binding metrics, where the subset is smaller than the set. For example, the subset may include a predefined number of clusters having the highest (or alternatively the lowest) cluster-specific binding metrics and/or is to include each cluster with a cluster-specific binding metric that exceeds a predefined threshold. In some instances, a number of clusters in the subset is based on how many aptamers are assigned to the clusters (e.g., such that the subset still includes the clusters having the highest cluster-specific binding metrics, but the subset is to include the smallest number of clusters that still results in a cumulative total count of aptamers assigned to the subset as being greater than an aptamer-count threshold).

In some instances, block 215 includes traversing the embedding space. The traversal may include gradient-ascent traversal or gradient-descent traversal (e.g., from a starting point that may randomly or pseudorandomly selected from across all projections or across all projections associate with a particular label. The traversal can be guided by labels assigned to aptamer representations along a traversal path. It will be appreciated that traversing the multi-dimensional space may corresponding to moving throughout the space in a manner that amounts to iteratively changing various bases.

In some instances, block 215 includes using interpolation (e.g., linear and/or spherical interpolation) to select data points (e.g., associated with labels that are less noisy than had been used to generate the embedding space). For example, a first select data point may be identified as one that corresponds to a low label (e.g., representing a predicted low binding affinity), and a second select data point may be identified as one that corresponds to a high label (e.g., representing a predicted high binding affinity). The first data point may correspond to a local or absolute extremum (e.g., maximum or minimum) of a label. The second data point may correspond to a local or absolute opposite extremum (e.g., minimum or maximum) of a label. A latent representation for the interpolation can be generated based on labels z at multiple coordinates (e.g., s₀ and s₁) to identify a potential point of interest (z_{α}). For example, a point of interest may be generated based on a correlation between and a variable of interest (e.g., binding affinity, functional inhibition, binding probability, etc.) or by using an interpolation technique (e.g., a spherical interpolation technique and/or a technique as disclosed by White, T. “Sampling Generative Networks”, available at https://arxiv.org/abs/1609.04468 (2016).

In some instances, block 215 includes using a support vector machine and labels generated by the Classification network to generate a decision boundary. The boundary can then be used to segregate representations corresponding to one type of prediction (e.g., high binding affinity) relative to another type of prediction (e.g., low binding affinity).

At block 220, for each embedding-space position of interest, a trained Decoder model is used to generate an aptamer sequence of interest. The trained Decoder model may have been trained in combination with the Encoder model used at block 210. Generating an aptamer sequence of interest may include generating a representation of interest in the embedding space, generating a one-hot representation of the aptamer sequence, generating a human-readable representation of the aptamer, etc. For example, block 220 may include transforming an embedding-space position into an input-space position (e.g., that may include one or more numbers or coordinates) and then using a conversion technique to generate an ordered set of base identifiers that uniquely and formulaically corresponds to the input-space position.

At block 225, an identification of the aptamer sequences of interest is output. For example, the identifications can be transmitted to a client device via a message controlling a presentation of a webpage or interface, the identifications can be included in a file that is availed or transmitted for presentation or download, or the identifications can be transmitted as an instruction file to a device configured to initiate a process to (for example) experimentally measure the binding affinity of each of one or more (or all) aptamers in the selected subset(s) and the particular binding target and/or experimentally measure whether each of one or more (or all) aptamers in the selected subset(s) binds to the particular binding target. In some instances, receipt of the instruction file from a device corresponding to a laboratory system may automatically trigger experiments to measure whether each aptamer in the subset binds to the particular binding target, to measure a binding affinity between each aptamer in the subset and the particular binding target, etc.

At least one, some or all of the aptamer sequences of interest may be used for further in vitro experimentation, for in vivo experimentation, and/or for clinical study. In some instances, the aptamer sequences identified at output are used in experiments (e.g., elution experiments) to measure a property (e.g., a binding affinity), and the sequences of interest are filtered down to one or more particular aptamer candidates for a given use.

For example, a clinical study may track the extent to which each of the one or more aptamer candidates effectively treats a given disease and/or results in undesirable side effects (e.g., via in vivo studies, mammalian studies, or human studies).

### Pipeline for Identifying and Experimentally Assessing Candidate Aptamers

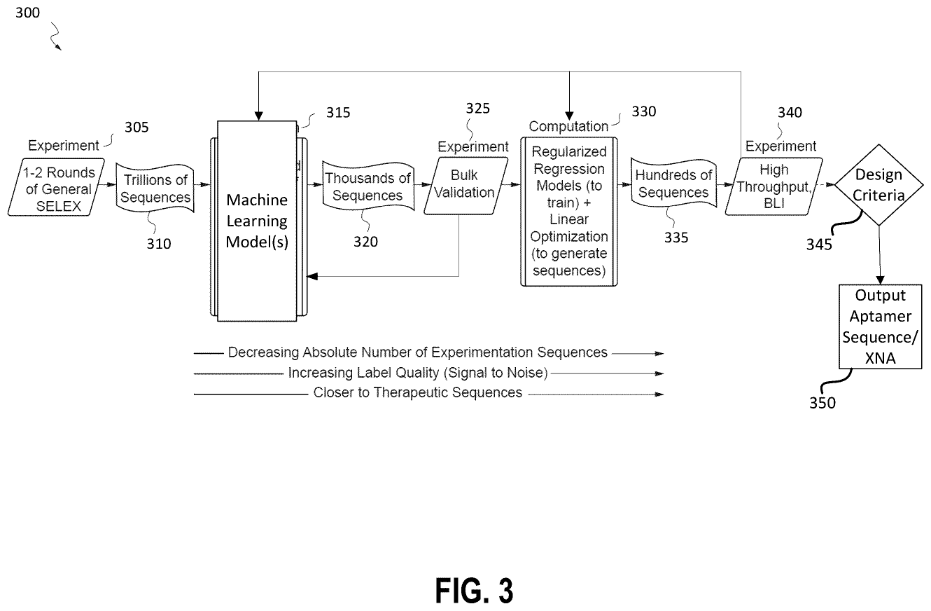

FIG. 3 shows a block diagram of a pipeline 300 for strategically identifying and generating high affinity binders of molecular targets. Pipeline 300 can include performing part or all of process 100 from FIG. 1, performing part of all of process 200 from FIG. 2 and/or performing one or more actions described herein. In various embodiments, the pipeline 300 implements in vitro experiments and in silico computation and machine-learning based techniques to iteratively improve a process for identifying binders (e.g., aptamers and/or binding regions) that can bind any given molecular target.

At block 305, in vitro binding selections (e.g., phages display or SELEX) are performed where a given molecular target (e.g., a protein of interest) is exposed to tens of trillions of different potential binders (e.g., a library of 10¹⁴-10¹⁵ nucleic acid aptamers), a separation protocol is used to remove non-binding aptamers (e.g., flow-through), and the binding aptamers are eluted from the given target. The binding aptamers and/or the non-binding aptamers are sequenced to predict which aptamers do and/or do not bind the given target. This binding selection process may be repeated for any number of cycles (e.g., 1 to 3 cycles) to reduce the absolute count of potential aptamers from tens of trillions of different potential aptamers down to millions or trillions of sequences 310 of aptamers identified to have some level of binding (specific and non-specific) for the given target.

In some instances, at least some of the millions or trillions of sequences 310 are labeled with one or more sequence properties. The one or more sequence properties may include a binding-approximation metric that indicates whether an aptamer included in or associated with the training data bound to a particular target. The binding-approximation metric can include (for example) a binary value or a categorical value. The binding-approximation metric can indicate whether the aptamer bound to the particular target in an environment where the aptamer and other aptamers (e.g., other potential aptamers) are concurrently introduced to the particular target. The binding-approximation metric can be determined using a high-throughput assay, such as in vitro binding selections (e.g., phages display or SELEX), a low-throughput assay, such as in vitro Bio-Layer Interferometry (BLI), or a combination thereof. Additionally or alternatively, the one or more sequence properties may include a function-approximation metric that indicates whether an aptamer included in or associated with the training data functions as intended (e.g., inhibits function A). The function-approximation metric can include (for example) a binary value or a categorical value. The function-approximation metric can be determined using a low-throughput assay, such as an optical fluorescence assay or any other assay capable of detecting function changes in a biological system (e.g., inhibiting an enzyme, inhibiting protein production, promoting binding between molecules, promoting transcription, etc.). Further, the function-approximation metric may be used to infer the binding-approximation metric (e.g., if function A is inhibited it can be inferred that the molecule bound to the particular target).

The sequences of binding aptamers, non-binding aptamers, or a combination thereof obtained from block 305 may have a low signal to noise ratio (and low label quality). In other words, the sequences in 310 may include a small amount of sequences of aptamers with specific binding or high affinity (signal) and a large amount of aptamer sequences with non-specific binding or low affinity binding to the given target (noise).

In some instances, at block 315, at least some of the sequences of binding aptamers, non-binding aptamers, or a combination thereof obtained from block 305 are used to train one or more Encoder models (e.g., where each of at least one of the one or more machine-learning models is a highly parameterized machine algorithm with a parameter count of greater than or equal to 10,000, 30,000, 50,000, or 75,000) and learn a fitness function capable of filtering, sorting, ranking, or otherwise evaluating the fitness (quality) of sequences of aptamers based on one or more constraints such as a design criteria proposed for an aptamer, a problem being solved (e.g., finding an aptamer that is capable of binding to target with a high-affinity), and/or an answer to a query (e.g., which aptamers are capable of inhibiting function A). In some instances, multiple Encoder models are trained to generate parallel and/or serial outputs. For example, Encoder models with different architectures, with different hyperparameters, having been trained using different loss functions, having been trained using different training-data subsets may be trained to generate outputs. For example, each Encoder model of one or more the Encoder model can include a model used to generate a projection at block 115 of process 100.

For each of the multiple Encoder models, an encoding efficiency η can be calculated. Further, a classification-metric metric c may be calculated using predictions from a corresponding Classifier model and a reconstruction error ∈ may be calculated based on predictions from a corresponding Decoder mode. A subset of the multiple models (e.g., a single model) may be selected based on the encoding efficiency η. The selection of the subset of the multiple models may further be based on the classification-metric metrics c and/or the reconstruction error ∈ associated with the multiple models.

Once the model is selected, the millions or trillions of sequences and their labels are fed to the Encoder model. It will be appreciated that, in some instances, only some of the sequences assessed by the clustering model may have a label. The labels of the some may be used to (for example) train the one or more models to generate sequence projections, and the trained models may then be used to generate projections for the sequences with labels and other sequences without labels.

The projections (e.g., and their corresponding labels) can then be used to identify a subset of the projected aptamers. The subset may include (for example) aptamers identified using a technique disclosed in relation to block 215 of process 200.

At block 325, identified sequences of aptamers 320 in the subset of clusters may be used to synthesize aptamers, which are used for subsequent binding selections. For example, subsequent in vitro binding selections (e.g., phages display or SELEX) may be performed where the given molecular target is exposed to the synthesized aptamers. A separation protocol may be used to remove non-binding aptamers (e.g., flow-through). The binding aptamers may then be eluted from the given target. The binding and/or non-binding aptamers may be sequenced to identify the sequence of aptamers that do and/or those that do not bind the given target. This binding selection process may be repeated for any number of cycles (e.g., 1 to 3 cycles) to validate which of the identified/designed aptamers from block 115 actually bind the given target. In some instances, the subsequent binding selections are performed using aptamers carrying Unique Molecular Identifiers (UMI) to enable accurate counting of copies of a given candidate sequence in elution or flow-through. Because the sequence diversity is reduced at this stage, there can be more copies of each aptamer to interact with the given target and improve the signal to noise ratio (and label quality).

The processes in blocks 305-325 may be performed once or repeated in part or in their entirety any number of times to decrease the absolute number of sequences and increase the signal to noise ratio, which ultimately results in a set of aptamer candidates that satisfy the one or more constraints (e.g., bind targets of interest in a inhibitory/activating fashion or to deliver a drug/therapeutic to a target such as a T-Cell). As used herein, “satisfy” the one or more constraints can be complete satisfaction (e.g., bound to the target), substantial satisfaction (e.g., bound to the target with an affinity above/below a given threshold or greater than 98% percent inhibition of a function A), or partial satisfaction (e.g., bound to the target at least 60% of the time or greater than 60% percent inhibition of a function A). The satisfaction of the constraint may be measured using one or more binding and/or analytical assays as described in detail herein.

The output from block 325 (e.g., bulk validation) may include aptamers that can bind to the target with varying strengths (e.g., high, medium or low affinities). The output from block 325 may also include aptamers that are not be capable of binding to the target. In some instances, the sequences of binding aptamers, non-binding aptamers, or a combination thereof obtained from block 325 are used to improve the machine-learning models in block 325 (e.g., by retraining or fine-tuning the machine-learning models that generate projections). The sequences of binding aptamers, non-binding aptamers, or a combination thereof from block 325 may be labeled with one or more sequence properties. The one or more sequence properties may include a binding metric that indicates whether an aptamer included in or associated with the training data bound to a particular target and/or a functional metric that indicates whether an aptamer included in or associated with the training data functions as intended (e.g., inhibits function A). In certain instances, the binding metric is determined from the subsequent in vitro binding selections (e.g., phages display or SELEX) performed in block 325 and/or a low-throughput assay, such as in vitro BLI.

At block 330, the sequences of binding aptamers, non-binding aptamers, or a combination thereof (optionally labeled with one or more sequence properties) obtained from block 325 are used to train an algorithm to identify sequences of aptamers 335 that can satisfy the one or more constraints (e.g., bind a given target). The algorithm may identify hundreds of additional or alternative sequences. The algorithm may include linear algorithms (for example) a support vector algorithm/machine or a regression algorithm (e.g., a linear regression algorithm). In some instances, the algorithm is a multiple regression algorithm. The regression algorithm may be trained using regularization techniques (i.e., fitting a model with more than one independent variable (covariates or predictors or features—all the same thing) to obtain a multiple regularized regression model. While the linear algorithms are less expressive than highly parametrized algorithms, the improved signal to noise at this stage can allow the linear algorithms to still capture signal while being better at generalizing.

Optimization techniques such as linear optimization may be used at this stage to identify the hundreds of additional or alternative sequences of aptamers 335 with differing relative fitness scores (and therefore affinity). Linear optimization (also called linear programming) is a computational method to achieve the best outcome (such as highest binding affinity for a given target) in a model whose requirements are represented by linear relationships (e.g., a regression model). More specifically, the linear optimization improves the linear objective function, subject to linear equality and linear inequality constraints to output the hundreds of additional or alternative sequences of aptamers 335 with differing relative fitness scores (including those with a highest binding affinity). Unlike the machine-learning model and searching process used in block 315, there may be greater confidence in deviating away from training data in the process of linear optimization due to better generalization by the regression models. Consequently, the linear optimization may not be constrained to a limited number of nucleotide edits away from the training dataset.

At block 340, identified or designed aptamer sequences 335 may be used to synthesize new aptamers. These new aptamers may then be characterized or validated using experiments 340. The experiments may include high throughput binding selections (e.g., SELEX) or low-throughput assays. In some instances, the low-throughput assay (e.g., BLI) is used to validate or measure a binding strength (e.g., affinity, avidity, or dissociation constant) of an aptamer to the given target. In this context, BLI may include preparing a biosensor tip to include the aptamers in an immobilized form and a solution with the given target in a tip of a biosensor. Binding between the molecule(s) and the particular target increases a thickness of the tip of the biosensor. The biosensor is illuminated using white light, and an interference pattern is detected. The interference pattern and temporal changes to the interference pattern (relative to a time at which the molecules and particular target are introduced to each other) are analyzed to predict binding-related characteristics, such as binding affinity, binding specificity, a rate of association, and a rate of dissociation. In other instances, the low-throughput assay (e.g., a spectrophotometer to measure protein concentration) is used to validate or measure functional aspects of the aptamer such as its ability to inhibit a biological function (e.g., protein production).

The processes in blocks 305-340 may be performed once or repeated in part or in their entirety any number of times to decrease the absolute number of sequences and increase the signal to noise ratio, which ultimately results in a set of aptamer candidates that best satisfy the one or more constraints (e.g., bind targets of interest in a inhibitory/activating fashion or to deliver a drug/therapeutic to a target such as a T-Cell). The output from block 140 (e.g., BLI) may include aptamers that can bind to the target with varying strengths (e.g., high, medium or low affinities). The output from block 340 may also include aptamers that are not be capable of binding to the target. In some instances, the sequences of binding aptamers, non-binding aptamers, or a combination thereof obtained from block 340 are used to improve the machine-learning models in block 315 and/or 330 (e.g., by retraining the machine-learning algorithms). The sequences of binding aptamers, non-binding aptamers, or a combination thereof from block 340 may be labeled with one or more sequence properties. As described herein, the one or more sequence properties may include a binding metric that indicates or predicts whether an aptamer included in or associated with the training data bound to a particular target and/or a functional metric that indicates or predicts whether an aptamer included in or associated with the training data functions as intended (e.g., inhibits function A). In certain instances, the binding-approximation metric is determined from the subsequent in vitro BLI performed in block 340.

In block 345, a determination is made as to whether one or more of the aptamers evaluated in block 340 satisfy the one or more constraints such as the design criteria proposed for an aptamer, the problem being solved (e.g., finding an aptamer that is capable of binding to target with a high-affinity), and/or the answer to a query (e.g., what aptamers are capable of inhibiting function A). The determination may be made based on the binding-approximation metric and/or the functional-approximation metric associated with an aptamer satisfying the one or more constraints. In some instances, an aptamer design criteria may be used to select one or more aptamers to be output as the final solution to the given problem. For example, the design criteria in block 345 may include a binding strength (e.g., a cutoff value), a minimum affinity or avidity between the aptamer and the target, or a maximum dissociation constant.

In block 350, one or more aptamers from experiments 340 that are determined to satisfy the one or more constraints (e.g., showing affinity greater or equal to the minimum cutoff) are provided, for example, as the final solution to the given problem or as a result to a given query. The providing the output may include generating an output library comprising the final set of aptamers. The output library may be generated incrementally as new aptamers are generated and selected by performing and/or repeating blocks 305-345. At each repetition cycle one or more aptamers may be identified (i.e., designed, generated and/or selected) and added to the output based on their ability to satisfy the one or more constraints. The providing the output may further include transmitting the one or more aptamers or output library to a user (e.g., transmitting electronically via wired or wireless communication).

It will be appreciated that although FIG. 3 and the description herein, describe going from trillions of sequences to thousands of sequences to hundreds of sequences, these numbers are merely provided for illustrative purposes. In general, it should be understood that pipeline 300 is provisioned to start with a large data set (a large absolute number of experimentation sequences which could be, for example, septillions, trillions, billions, or millions) for training a highly-parametrized algorithm and eventually narrows down the absolute number of experimentation sequences to a more manageable number eventually aligning on a small data set (a small absolute number of experimentation sequences which could be, for example, hundreds, tens, or less) for low-throughput characterization and validation as potential therapeutic candidates.

It will further be appreciated that process 300 need not be completed in its entirety. For example, some or all of blocks 325-345 may omitted from process 300. To illustrate, the machine-learning model(s) may be used to identify candidate aptamers to experimentally investigate for a given aim (e.g., binding to a given target), and identification of the candidate aptamers may be transmitted to a client (who may then experimentally test some or all of the candidate aptamers).

### EXAMPLE

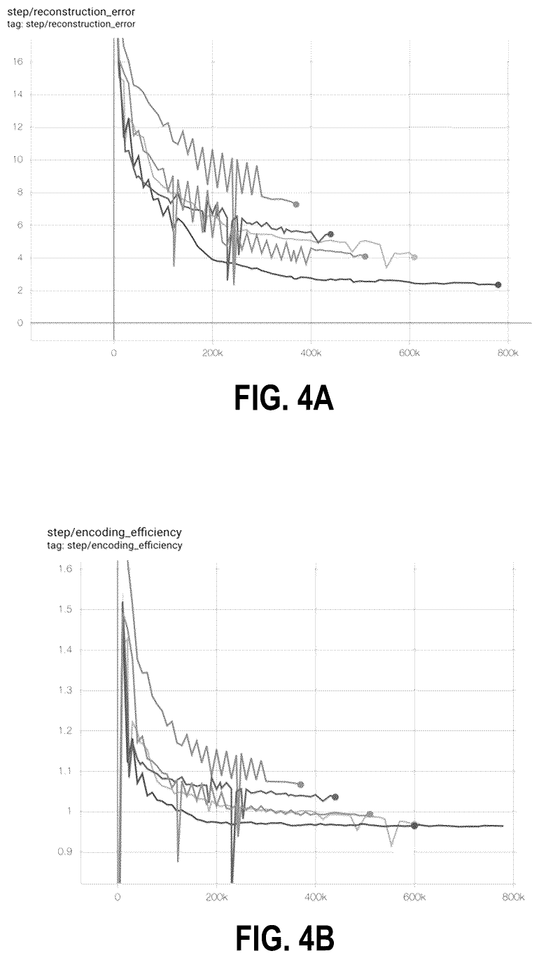

Five variational autoencoder (VAE) models were configured with different sets of hyperparameters and trained to identify functionally inhibiting Trypsin TNA aptamers. FIGS. 4A and 4B show the reconstruction error and encoding efficiency (respectively) for each of the five models. The x-axis is the number of gradient descent steps performed on the model, and the y-axis is the respective metric value at that point in training. This data indicates that, over the course of training, each model became better at reconstructing the aptamer sequences (given that all curves in FIG. 4A trend downwards). However, certain models converge to a superior degree of distribution compression as compared to other models, as indicated by the higher encoding efficiency represented in FIG. 4B as the curves flatten out at around 300,000 steps.

Further, there appears a trade-off between encoding efficiency and reconstruction error: models which converge to the smallest reconstruction error (e.g. red) tend to have the lowest encoding efficiency. Models with encoding efficiency of less than 1 encode at least as much information in the latent space as is in the original sequence space, indicating that it is unlikely that they have identified any high-level structure or properties of the sequence distribution of the training data.

The green curve indicates that the corresponding model failed to achieve a reconstruction error of less than 5 but does achieve an encoding efficiency of greater than 1, indicating that it has successfully arrived at a distributional compression of the sequence space.

This example indicates that a selection of a model based only on reconstruction error (and not considering encoding efficiency) may result in selecting a model that has a relatively poor (<1) encoding efficiency. However, when encoding efficiency is also considered, a superior model (e.g., corresponding to the green curve) can be selected that still has relatively small reconstruction error while also achieving distribution compression (corresponding to an encoding efficiency of greater than 1).

Some embodiments of the present disclosure include a system including one or more data processors. In some embodiments, the system includes a non-transitory computer readable storage medium containing instructions which, when executed on the one or more data processors, cause the one or more data processors to perform part or all of one or more methods and/or part or all of one or more processes disclosed herein. Some embodiments of the present disclosure include a computer-program product tangibly embodied in a non-transitory machine-readable storage medium, including instructions configured to cause one or more data processors to perform part or all of one or more methods and/or part or all of one or more processes disclosed herein.

The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention as claimed has been specifically disclosed by embodiments and optional features, modification, and variation of the concepts herein disclosed may be resorted to by those skilled in the art, and that such modifications and variations are considered to be within the scope of this invention as defined by the appended claims.

The description provides preferred exemplary embodiments only, and is not intended to limit the scope, applicability or configuration of the disclosure. Rather, the description of the preferred exemplary embodiments will provide those skilled in the art with an enabling description for implementing various embodiments. It is understood that various changes may be made in the function and arrangement of elements without departing from the spirit and scope as set forth in the appended claims.

Specific details are given in the following description to provide a thorough understanding of the embodiments. However, it will be understood that the embodiments may be practiced without these specific details. For example, circuits, systems, networks, processes, and other components may be shown as components in block diagram form in order not to obscure the embodiments in unnecessary detail. In other instances, well-known circuits, processes, algorithms, structures, and techniques may be shown without unnecessary detail in order to avoid obscuring the embodiments.

What is claimed is:

1. A computer-implemented method comprising:
accessing a multi-dimensional latent space that corresponds to projections of sequences of aptamers, wherein the multi-dimensional latent space was defined by an Encoder model, wherein an architecture of the Encoder model, at least one hyperparameter of the Encoder model, or at least one characteristic of a training data set used to train the Encoder model was selected using an assessment of an encoding-efficiency of the Encoder model that is based on:
a predicted extent to which representations in an embedding space are indicative of specific aptamer sequences; and
a predicted extent to which a probability distribution of the embedding space differs from a probability distribution of a source space, wherein the source space represents individual base-pairs;
generating a set of projections in the multi-dimensional latent space using representations of a plurality of aptamers and the Encoder model;
identifying one or more candidate aptamers for the particular target using the set of projections and using the Decoder model, wherein the one or more candidate aptamers are a subset of the plurality of aptamers; and
outputting an identification of the one or more candidate aptamers.

2. The computer-implemented method of claim 1, wherein the selection of the architecture of the Encoder network, the at least one hyperparameter of the Encoder network, or the at least one characteristic of the training data set used to train the Encoder network was further based on a classification-performance metric corresponding to predictions of a Classifier model when different architectures, hyperparameters, or training sets were used to configure or train the Encoder network.

3. The computer-implemented method of claim 1, wherein the extent to which a probability distribution of the embedding space differs from a probability distribution of a source space includes a Kullback-Leibler distance.

4. The computer-implemented method of claim 1, wherein the extent to which representations in an embedding space are indicative of specific aptamer sequences is based on a reconstruction error relative to predictions of the Decoder model when different architectures, hyperparameters, or training sets were used to configure or train the Encoder network.

5. The computer-implemented method of claim 1, further comprising, prior to accessing the multi-dimensional latent space:
selecting the architecture of the Encoder model based on:
the predicted extent to which representations in an embedding space are indicative of specific aptamer sequences; and
the predicted extent to which a probability distribution of the embedding space differs from the probability distribution of a source space.

6. The computer-implemented method of claim 1, further comprising, prior to accessing the multi-dimensional latent space:
selecting the at least one hyperparameter of the Encoder model based on:
the predicted extent to which representations in an embedding space are indicative of specific aptamer sequences; and
the predicted extent to which a probability distribution of the embedding space differs from the probability distribution of a source space.

7. The computer-implemented method of claim 1, further comprising, prior to accessing the multi-dimensional latent space:
selecting the at least one characteristic of a training data set of the Encoder model based on:
the predicted extent to which representations in an embedding space are indicative of specific aptamer sequences; and
the predicted extent to which a probability distribution of the embedding space differs from the probability distribution of a source space.

8. The computer-implemented method of claim 1, wherein the architecture of the Encoder network was selected using the assessment of the encoding efficiency.

9. The computer-implemented method of claim 1, wherein the at least one hyperparameter of the Encoder network was selected using the assessment of the encoding efficiency.

10. The computer-implemented method of claim 1, wherein the at least one characteristic of the training data set of the Encoder network was selected using the assessment of the encoding efficiency.

11. A system comprising:
one or more data processors; and
a non-transitory computer readable storage medium containing instructions which, when executed on the one or more data processors, cause the one or more data processors to perform a set of actions including:
accessing a multi-dimensional latent space that corresponds to projections of sequences of aptamers, wherein the multi-dimensional latent space was defined by an Encoder model, wherein an architecture of the Encoder model, at least one hyperparameter of the Encoder model, or at least one characteristic of a training data set used to train the Encoder model was selected using an assessment of an encoding-efficiency of the Encoder model that is based on:
a predicted extent to which representations in an embedding space are indicative of specific aptamer sequences; and
a predicted extent to which a probability distribution of the embedding space differs from a probability distribution of a source space, wherein the source space represents individual base-pairs;
generating a set of projections in the multi-dimensional latent space using representations of a plurality of aptamers and the Encoder model;
identifying one or more candidate aptamers for the particular target using the set of projections and using the Decoder model, wherein the one or more candidate aptamers are a subset of the plurality of aptamers; and
outputting an identification of the one or more candidate aptamers.

12. The system of claim 11, wherein the selection of the architecture of the Encoder network, the at least one hyperparameter of the Encoder network, or the at least one characteristic of the training data set used to train the Encoder network was further based on a classification-performance metric corresponding to predictions of a Classifier model when different architectures, hyperparameters, or training sets were used to configure or train the Encoder network.

13. The system of claim 11, wherein the extent to which a probability distribution of the embedding space differs from a probability distribution of a source space includes a Kullback-Leibler distance.

14. The system of claim 11, wherein the extent to which representations in an embedding space are indicative of specific aptamer sequences is based on a reconstruction error relative to predictions of the Decoder model when different architectures, hyperparameters, or training sets were used to configure or train the Encoder network.

15. The system of claim 11, wherein the set of actions further includes, prior to accessing the multi-dimensional latent space:
selecting the architecture of the Encoder model based on:
the predicted extent to which representations in an embedding space are indicative of specific aptamer sequences; and
the predicted extent to which a probability distribution of the embedding space differs from the probability distribution of a source space.

16. The system of claim 11, wherein the set of actions further includes, prior to accessing the multi-dimensional latent space:
selecting the at least one hyperparameter of the Encoder model based on:
the predicted extent to which representations in an embedding space are indicative of specific aptamer sequences; and
the predicted extent to which a probability distribution of the embedding space differs from the probability distribution of a source space.

17. The system of claim 11, wherein the set of actions further includes, prior to accessing the multi-dimensional latent space:
selecting the at least one characteristic of a training data set of the Encoder model based on:
the predicted extent to which representations in an embedding space are indicative of specific aptamer sequences; and
the predicted extent to which a probability distribution of the embedding space differs from the probability distribution of a source space.

18. A computer-program product tangibly embodied in a non-transitory machine-readable storage medium, including instructions configured to cause one or more data processors to perform a set of actions including:
accessing a multi-dimensional latent space that corresponds to projections of sequences of aptamers, wherein the multi-dimensional latent space was defined by an Encoder model, wherein an architecture of the Encoder model, at least one hyperparameter of the Encoder model, or at least one characteristic of a training data set used to train the Encoder model was selected using an assessment of an encoding-efficiency of the Encoder model that is based on:
a predicted extent to which representations in an embedding space are indicative of specific aptamer sequences; and
a predicted extent to which a probability distribution of the embedding space differs from a probability distribution of a source space, wherein the source space represents individual base-pairs;
generating a set of projections in the multi-dimensional latent space using representations of a plurality of aptamers and the Encoder model;
identifying one or more candidate aptamers for the particular target using the set of projections and using the Decoder model, wherein the one or more candidate aptamers are a subset of the plurality of aptamers; and
outputting an identification of the one or more candidate aptamers.

19. The computer-program product of claim 18, wherein the selection of the architecture of the Encoder network, the at least one hyperparameter of the Encoder network, or the at least one characteristic of the training data set used to train the Encoder network was further based on a classification-performance metric corresponding to predictions of a Classifier model when different architectures, hyperparameters, or training sets were used to configure or train the Encoder network.

20. The computer-program product of claim 18, wherein the extent to which a probability distribution of the embedding space differs from a probability distribution of a source space includes a Kullback-Leibler distance.
